(19) United States
(12) Patent Application Publication
(10) Pub. No.: US 2013/0338228 A1
(43) Pub. Date: Dec. 19, 2013

(54) METHODS FOR FACILITATING MUSCLE RECOVERY AFTER A PERIOD OF DISUSE USING BETA-HYDROXY-BETA-METHYLBUTYRATE

(71) Applicants: Suzette L. Pereira, Westerville, OH (US); Neile K. Edens, Columbus, OH (US)

(72) Inventors: Suzette L. Pereira, Westerville, OH (US); Neile K. Edens, Columbus, OH (US)

(73) Assignee: ABBOTT LABORATORIES, ABBOTT PARK, IL (US)

(21) Appl. No.: 13/990,726

(22) PCT Filed: Dec. 20, 2011
(86) PCT No.: PCT/US2011/066258

§ 371 Date: Sep. 5, 2013

Related U.S. Application Data
(60) Provisional application No. 61/427,249, filed on Dec. 27, 2010.

(51) Int. Cl. A61K 31/19

(52) U.S. Cl. CPC A61K 31/19
USPC 514/557

(57) ABSTRACT

Disclosed are methods utilizing beta-hydroxy-beta-methylbutyrate (HMB) for facilitating the recovery of muscle after a period of muscle disuse. The HMB facilitates the recovery of muscle mass in an individual and can also be used to prevent further muscle atrophy typically associated with muscle reloading after extended periods of muscle disuse in the individual. The methods disclosed may be particularly suitable for older adults.

### FIELD OF THE DISCLOSURE

The present disclosure relates to methods for facilitating the recovery of muscle after a period of muscle disuse and/or muscle inactivity using nutritional compositions comprising beta-hydroxy-beta-methylbutyrate (HMB).

### BACKGROUND OF THE DISCLOSURE

Prolonged muscle disuse or immobilization due to bed rest, hospitalization, casting, and the like, can cause rapid muscle atrophy and muscle force loss. The atrophy and muscle force loss is generally exacerbated with aging. Animal studies have suggested that the recovery of muscle mass and muscle force following a period of extended disuse is a slow and difficult process, and may be incomplete in aged muscles.

Loss of muscle mass, function, and force can impair mobility and may increase the susceptibility of an individual to further muscle or other injury. In older adults in particular, this may lead to a decrease in the independence and quality of life.

Conventionally, exercise during the recovery/reloading period after muscle disuse has been the only intervention that has provided some muscle rebuilding benefits. Exercise, however, may not always be a feasible alternative, especially if a patient is elderly and/or recovering from severe illness or surgery. To date, nutritional interventions that can hasten muscle recovery after periods of extensive inactivity or immobilization have not been available.

As such, it would be desirable to formulate nutritional compositions and methods for using the compositions that could be used to effectively facilitate muscle recovery and reloading after a period of muscle disuse, inactivity or immobilization. It would also be beneficial if the nutritional compositions and methods could be used to build muscle mass and improve muscle force in individuals that are not able to exercise. Additionally, it would be beneficial if the nutritional compositions and methods could prevent further muscle atrophy during the rebuilding of muscle mass after extended disuse.

### SUMMARY OF THE DISCLOSURE

The present disclosure is directed generally to methods of facilitating muscle recovery in an individual after a period of muscle disuse. The individual may have muscle atrophy due to the muscle disuse, and the methods of the present disclosure can reduce the time period required to re-build muscle after muscle atrophy. The methods of facilitating muscle recovery utilize nutritional compositions including beta-hydroxy-beta methylbutyrate. Some embodiments of the present disclosure may be particularly suitable for adults, including older adults, who may have particular difficulty recovering from significant muscle loss and muscle atrophy.

One embodiment is directed to a method for facilitating muscle recovery in an individual having muscle atrophy caused by a period of muscle disuse. The method comprises administering to the individual during the period of muscle disuse and during a period of muscle recovery a composition comprising an effective amount of beta-hydroxy-beta-methylbutyrate.

Another embodiment is directed to a method for minimizing muscle atrophy in an individual whose muscles have been subject to a period of muscle disuse. The method comprises administering to the individual during the period of muscle disuse and during a period of muscle recovery a composition comprising an effective amount of beta-hydroxy-beta-methylbutyrate.

Another embodiment is directed to a method for facilitating muscle recovery in an older adult having muscle atrophy caused by a period of muscle disuse. The method comprises administering to the older adult during the period of muscle disuse and during a period of muscle recovery a composition comprising an effective amount of beta-hydroxy-beta-methylbutyrate.

It has been found that beta-hydroxy-beta-methylbutyrate (HMB) can be administered to an individual to minimize muscle atrophy resulting from muscle disuse, such as disuse caused by being in a cast for an extended period of time, and facilitate the recovery of muscle in an individual. By administering to the individual HMB during the period of disuse and during the period of recovery, muscle mass and/or muscle force of the individual is enhanced. Surprisingly, it has been found that HMB stimulates muscle protein synthesis during recovery/reloading periods (i.e., after muscles are remobilized), in the absence of muscle insult and in the absence of exercise.

Accordingly, the methods of the present disclosure offer an alternative therapeutic option that may contribute to the recovery of healthy muscle mass and force in individuals that have been subjected to muscle disuse. These benefits are advantageously achieved without the need for a strenuous exercise routine, and may be particularly beneficial in older adults.

### BRIEF DESCRIPTION OF THE FIGURES

FIG. 1 is a graph depicting the change in body weight in aged animals after a period of disuse as evaluated in Example 1.

FIG. 2 is a graph depicting the change in muscle force in aged animals after a period of disuse as evaluated in Example 1.

FIGS. 3A and 3B depict the change in muscle weight for plantaris muscle and soleus muscle in aged animals after a period of disuse as evaluated in Example 1.

FIGS. 4A and 4B depict the change in muscle fiber cross-section for plantaris muscle and soleus muscle in aged animals after a period of disuse as evaluated in Example 1.

FIGS. 4C and 4D depict the change in muscle fiber frequency distribution for plantaris muscle and soleus muscle in aged animals after a period of disuse as evaluated in Example 1.

FIG. 5 is a graph depicting the frequency of TUNEL positive myonuclei in plantaris muscle as evaluated in Example 1.

FIG. 6 is a graph depicting the frequency of TUNEL positive myonuclei in soleus muscle as evaluated in Example 1.

FIGS. 7A and 7B depict Bax protein content in plantaris and soleus muscles after hind limb suspension and reloading as evaluated in Example 1.

FIGS. 8A and 8B depict Cleaved Caspase-9 protein content in plantaris and soleus muscles after hind limb suspension and reloading as evaluated in Example 1.

FIGS. 9A and 9B depict Cleaved Caspase-3 protein content in plantaris and soleus muscles after hind limb suspension and reloading as evaluated in Example 1.

FIGS. 10A and 10B depict Bcl-2 protein content in plantaris and soleus muscles after hind limb suspension and reloading as evaluated in Example 1.

FIG. 11 depicts the activation of satellite cells by HMB using BrdU labeling at 14 days reloading.

### DETAILED DESCRIPTION OF THE DISCLOSURE

The methods for muscle recovery after a period of muscle disuse of the present disclosure utilize beta-hydroxy-beta methylbutyrate (HMB) to facilitate muscle recovery in an individual having muscle atrophy caused by muscle disuse. The features of the methods, as well as some of the many optional variations and additions, are described in detail hereafter.

The term “calcium HMB” as used herein, unless otherwise specified, refers to the calcium salt of beta-hydroxy-beta-methylbutyrate (also referred to as beta-hydroxyl-3-methyl butyric acid, beta-hydroxy-beta methylbutyric acid), beta-hydroxy isovaleric acid, or HMB), which is most typically in a monohydrate form. All weights, percentages, and concentrations as used herein to characterize calcium HMB are based on the weight of calcium HMB monohydrate, unless otherwise specified.

The term “nutritional product” as used herein, unless otherwise specified, refers to nutritional liquids, nutritional powders, nutritional semi-solids, and nutritional semi-liquids, some of which may be reconstituted to form a nutritional liquid, and are suitable for oral consumption by a human.

The term “muscle recovery” as used herein, unless otherwise specified, refers to an increase in muscle mass and/or muscle force.

The term “period of muscle disuse” as used herein, unless otherwise specified, refers to a period of muscle inactivity, including extended muscle inactivity, or full or partial immobilization of a body muscle resulting from bed rest, hospitalization, casting, and the like. In one specific embodiment, “period of muscle disuse” includes muscles in the arms or legs that have suffered from disuse, including extended disuse.

The term “extended” when referencing “extended inactivity” or “extended disuse” as used herein, unless otherwise specified, refers to inactivity or full or partial immobilization of a body muscle resulting from bed rest, hospitalization, casting, and the like for a time period of at least 1 week, including at least 4 weeks, including at least 6 weeks, including at least 2 months, including at least 6 months, and including 1 year or more.

The term “period of muscle recovery” as used herein, unless otherwise specified, refers to the period of time after the muscle disuse has ended and use and recovery of the muscle begins.

The term “facilitating” as used herein, unless otherwise specified, refers to aiding or assisting or helping such that “facilitating muscle recovery” refers to aiding in muscle recovery or assisting in muscle recovery or helping in muscle recovery.

The term “period” as used herein, unless otherwise specified, refers to a unit of time such that “period of muscle disuse” refers to a unit of time in which muscle disuse occurred.

The term “older adult” as used herein, unless otherwise specified, refers to an adult at least 55 years of age, including from 55 to about 85 years of age.

All percentages, parts and ratios as used herein, are by weight of the total product, unless otherwise specified. All such weights as they pertain to listed ingredients are based on the active level and, therefore, do not include solvents or by-products that may be included in commercially available materials, unless otherwise specified.

All references to singular characteristics or limitations of the present disclosure shall include the corresponding plural characteristic or limitation, and vice versa, unless otherwise specified or clearly implied to the contrary by the context in which the reference is made.

All combinations of method or process steps as used herein can be performed in any order, unless otherwise specified or clearly implied to the contrary by the context in which the referenced combination is made.

The various embodiments of the nutritional products used in the methods of the present disclosure may also be substantially free of any optional or selected essential ingredient or feature described herein, provided that the remaining composition still contains all of the required ingredients or features as described herein. In this context, and unless otherwise specified, the term “substantially free” means that the selected product contains less than a functional amount of the optional ingredient, typically less than about 1%, including less than about 0.5%, including less than about 0.1%, and also including zero percent, by weight of such optional or selected essential ingredient.

The nutritional products and methods may comprise, consist of, or consist essentially of the essential elements of the products as described herein, as well as any additional or optional element described herein or otherwise useful in nutritional product applications.

### Product Form

The nutritional products including the HMB useful in the methods of the present disclosure may be formulated in any known or otherwise suitable product form for oral or parenteral administration. Oral product forms are generally preferred and include any solid, semi-solid, liquid, semi-liquid, or powder formulation suitable for use herein, provided that such a formulation allows for safe and effective oral delivery of the essential and other selected ingredients from the selected product form.

Non-limiting examples of solid nutritional product forms suitable for use in the methods herein include snack and meal replacement products, including those formulated as bars, sticks, cookies or breads or cakes or other baked goods, frozen liquids, candy, breakfast cereals, powders or granulated solids or other particulates, molded or compressed powders, snack chips or bites, frozen or retorted entrees and so forth.

Non-limiting examples of liquid product forms suitable for use herein include snack and meal replacement products, hot or cold beverages, carbonated or non carbonated beverages, juices or other acidified beverages, milk or soy-based beverages, shakes, coffees, teas, enteral feeding compositions, and so forth. These liquid compositions are most typically formulated as suspensions or emulsions, but can also be formulated in any other suitable forms such as clear liquids, substantially clear liquids, solutions, and so forth.

As noted above, the nutritional products may be in the form of a semi-solid, which includes those forms that are intermediate in properties, such as rigidity, between solids and liquids. Some semi-solids examples include puddings, gelatins, and doughs.

Additionally as noted, the nutritional products may be in the form of a semi-liquid, which includes those forms that are intermediate in properties, such as flow properties, between liquids and solids. Exemplary semi-liquids include thick shakes and liquid gels.

Other non-limiting examples of suitable oral product forms include conventional product forms such as capsules, tablets, caplets, pills, and so forth.

The quantity of the nutritional product for providing an effective amount of HMB to the targeted user may be contained in one or a plurality of individual dosage forms that may be administered in single or multiple dosages per day.

The nutritional products including HMB may be formulated with sufficient kinds and amounts of nutrients to provide a sole, primary, or supplemental source of nutrition, or to provide a specialized nutritional product for use in individuals afflicted with specific diseases or conditions or with a targeted nutritional benefit. In many embodiments, the nutritional product will include protein, fat, and carbohydrate in addition to the HMB.

### Beta-Hydroxy-Beta-Methylbutyrate (HMB)

The nutritional products comprise HMB, which means that the products are either formulated with the addition of HMB, most typically as a calcium monohydrate, or are otherwise prepared so as to contain HMB in the finished product. Any source of HMB is suitable for use herein provided that the finished product contains HMB, although such a source is preferably calcium HMB and is most typically added as such to the nutritional products during formulation.

Although calcium HMB monohydrate is the preferred source of HMB for use herein, other suitable sources may include HMB as a free acid, a salt, an anhydrous salt, an ester, a lactone, or other product forms that otherwise provide a bioavailable form of HMB from the nutritional product. Non-limiting examples of suitable salts of HMB for use herein include HMB salts, hydrated or anhydrous, of sodium, potassium, magnesium, chromium, calcium, or other non-toxic salt form. Calcium HMB monohydrate is preferred and is commercially available from Technical Sourcing International (TSI) of Salt Lake City, Utah and from Lonza Group Ltd. (Basel, Switzerland).

When the nutritional product is a liquid, the effective concentration of HMB in the liquid may range up to about 10%, including from about 0.01% to about 10%, and also including from about 0.1% to about 5.0%, and also including from about 0.3% to about 2%, and also including from about 0.4% to about 1.5%, and also including from about 0.3% to about 0.6% by weight of the nutritional liquid.

When the nutritional product is a solid, the effective concentration of HMB in the solid may range up to about 10%, including from about 0.1% to about 8%, and also including from about 0.2% to about 5.0%, and also including from about 0.3% to about 3%, and also including from about 0.3% to about 1.5%, and also including from about 0.3% to about 0.6% by weight of the nutritional powder.

The nutritional products may provide from about 0.1 to about 10 grams/day of HMB in accordance with the methods described herein. Accordingly, the nutritional products may provide from about 0.1 to about 10 grams, including from about 0.5 to about 5.0 grams, including from about 0.5 to about 2.5 grams, including from about 1.0 to about 1.7 grams, including about 1.5 grams of HMB per serving, wherein an exemplary serving may be about 240 ml of ready to feed nutritional liquid or about 240 ml of reconstituted nutritional solid. An individual may be administered one serving per day, two servings per day, three servings per day, or four or more servings per day to receive the desired amount of HMB from the nutritional product.

### Macronutrients

The nutritional products may further comprise one or more optional macronutrients in addition to the HMB described herein. The optional macronutrients include proteins, lipids, carbohydrates, and combinations thereof. In some embodiments, the nutritional products are desirably formulated as nutritional liquids containing all three macronutrients in addition to the HMB.

Macronutrients suitable for use herein include any protein, lipid, or carbohydrate or source thereof that is known for, or otherwise suitable for, use in an oral nutritional product, provided that the optional macronutrient is safe and effective for oral administration and is otherwise compatible with the other ingredients in the nutritional product.

The concentration or amount of optional lipid, carbohydrate, and protein in the nutritional product can vary considerably depending upon the particular nutritional application of the product. These optional macronutrients are most typically formulated within any of the embodied ranges described in the following tables.

| Nutrient (% total calories) | Example A | Example B | Example C |
|---|---|---|---|
| Carbohydrate | 0-100 | 10-70 | 40-50 |
| Lipid | 0-100 | 20-65 | 35-55 |
| Protein | 0-100 | 5-40 | 15-25 |

Each numerical value preceded by the term “about”

| Nutrient (wt % composition) | Example D | Example E | Example F |
|---|---|---|---|
| Carbohydrate | 0-98 | 1-50 | 10-30 |
| Lipid | 0-98 | 1-30 | 3-15 |
| Protein | 0-98 | 1-30 | 2-10 |

Each numerical value preceded by the term “about”

### Carbohydrate

Optional carbohydrates suitable for use in the nutritional products may be simple, complex, or variations or combinations thereof, all of which are optionally in addition to the HMB as described herein. Non-limiting examples of suitable carbohydrates include hydrolyzed or modified starch or cornstarch, maltodextrin, isomaltulose, sucromalt, glucose polymers, sucrose, corn syrup, corn syrup solids, rice-derived carbohydrate, glucose, fructose, lactose, high fructose corn syrup, honey, sugar alcohols (e.g., maltitol, erythritol, sorbitol), and combinations thereof.

Optional carbohydrates suitable for use in the nutritional products also include soluble dietary fiber, non-limiting examples of which include gum Arabic, fructooligosaccharide (FOS), sodium carboxymethyl cellulose, guar gum, citrus pectin, low and high methoxy pectin, oat and barley glucans, carrageenan, psyllium and combinations thereof. Insoluble dietary fiber is also suitable as a carbohydrate source herein, non-limiting examples of which include oat hull fiber, pea hull fiber, soy hull fiber, soy cotyledon fiber, sugar beet fiber, cellulose, corn bran, and combinations thereof. In one specific embodiment, the carbohydrate system includes a combination of carbohydrate sources including maltodextrin (optionally low DE maltodextrin) and sucrose.

In some specific embodiments, the concentration of carbohydrate in liquid nutritional embodiments may range from about 5.0% to about 40%, including from about 7.0% to about 30%, and also including from about 10% to about 25%, and also including about 10.2%, by weight of the liquid nutritional.

In other specific embodiments, the concentration of carbohydrate in powder embodiments may range from about 10% to about 90%, including from about 20% to about 80%, and also including from about 40% to about 60%, by weight of the nutritional powder. In one specific embodiment, the carbohydrate is present in the nutritional powder in an amount of about 58%, by weight of the nutritional powder.

### Protein

Optional proteins suitable for use in the nutritional products include hydrolyzed, partially hydrolyzed or non-hydrolyzed proteins or protein sources, and can be derived from any known or otherwise suitable source such as milk (e.g., casein, whey), animal (e.g., meat, fish, egg albumen), cereal (e.g., rice, corn), vegetable (e.g., soy, pea, potato), or combinations thereof. The proteins for use herein can also include, or be entirely or partially replaced by, free amino acids known for use in nutritional products, non-limiting examples of which include L-tryptophan, L-glutamine, L-tyrosine, L-methionine, L-cysteine, taurine, L-arginine, L-carnitine, and combinations thereof.

In some specific embodiments, the concentration of protein in liquid nutritional embodiments may range from about 1.0% to about 30%, including from about 1.0% to about 15%, and also including from about 1.0% to about 10%, and also including from about 1.0% to about 7.0%, by weight of the liquid nutritional.

In other specific embodiments, the concentration of protein in powder embodiments may range from about 1.0% to about 50%, including from about 10% to about 50%, and also including from about 10% to about 30%, by weight of the nutritional powder.

In one specific embodiment, the protein system includes a combination of protein sources including calcium (or sodium) caseinate and soy protein isolate. In another specific embodiment, the protein system includes a combination of protein sources including sodium (or calcium) caseinate, milk protein concentrate, soy protein isolate, and whey protein concentrate.

### Lipid

Optional lipids suitable for use in the nutritional products include coconut oil, fractionated coconut oil, soy oil, corn oil, olive oil, safflower oil, high oleic safflower oil, high GLA-safflower oil, MCT oil (medium chain triglycerides), sunflower oil, high oleic sunflower oil, palm and palm kernel oils, palm olein, canola oil, flaxseed oil, borage oil, soybean oil, cottonseed oils, evening primrose oil, blackcurrant seed oil, transgenic oil sources, fungal oils, marine oils (e.g., tuna, sardine) and so forth. In one specific embodiment, the fat system includes a combination of fat sources including a high oleic safflower oil, canola oil, and soy oil.

In some specific embodiments, the concentration of lipid in liquid nutritional embodiments may range from about 1.0% to about 30%, including from about 1.0% to about 20%, and also including from about 1.0% to about 15%, and also including from about 1.5% to about 5.0%, by weight of the liquid nutritional. In one specific embodiment, the nutritional liquid includes lipid in an amount of about 1.6%, by weight of the nutritional liquid.

In other specific embodiments, the concentration of lipid in powder embodiments may range from about 1.0% to about 30%, including from about 1.0% to about 20%, and also including from about 1.0% to about 15%, and also including from about 5.0% to about 10%, by weight of the nutritional powder. In one specific embodiment, the nutritional powder includes lipid in an amount of about 7.5%, by weight of the nutritional powder.

### Optional Ingredients

The nutritional products comprising HMB and optionally one or more macronutrients may further comprise other optional ingredients that may modify the physical, nutritional, chemical, hedonic or processing characteristics of the products or serve as pharmaceutical or additional nutritional components when used in a targeted population. Many such optional ingredients known or otherwise suitable for use in other nutritional products may also be used in the nutritional products described herein, provided that such optional ingredients are safe and effective for oral administration and are compatible with the essential and other ingredients in the selected product form.

Non-limiting examples of such optional ingredients include preservatives, antioxidants, beta-alanine, emulsifying agents, buffers, pharmaceutical actives, additional nutrients as described herein, colorants, flavors, thickening agents and stabilizers, and so forth.

The nutritional products may further comprise vitamins or related nutrients, non-limiting examples of which include vitamin A, vitamin D, vitamin E, vitamin K, thiamine, riboflavin, pyridoxine, vitamin B 12, carotenoids, niacin, folic acid, pantothenic acid, biotin, vitamin C, choline, inositol, salts, and derivatives thereof, and combinations thereof.

The nutritional products may further comprise additional minerals, non-limiting examples of which include phosphorus, magnesium, calcium, sodium, potassium, molybdenum, chromium, selenium, chloride, and combinations thereof.

The nutritional products may also include one or more flavoring or masking agents. Suitable flavoring or masking agents include natural and artificial sweeteners, sodium sources such as sodium chloride, and hydrocolloids, such as guar gum, xanthan gum, carrageenan, gellan gum, gum acacia and combinations thereof.

### Methods of Manufacture

The nutritional products may be manufactured by any known or otherwise suitable method for making nutritional products including nutritional liquids such as emulsions.

In one suitable manufacturing process, a nutritional liquid is prepared using at least three separate slurries, including a protein-in-fat (PIF) slurry, a carbohydrate-mineral (CHO-MIN) slurry, and a protein-in-water (PIW) slurry. The PIF slurry is formed by heating and mixing the selected oils (e.g., canola oil, corn oil, fish oil, etc.) and then adding an emulsifier (e.g., lecithin), fat soluble vitamins, and a portion of the total protein (e.g., milk protein concentrate, etc.) with continued heat and agitation. The CHO-MIN slurry is formed by adding with heated agitation to water: minerals (e.g., potassium citrate, dipotassium phosphate, sodium citrate, etc.), trace and ultra trace minerals (TM/UTM premix), thickening or suspending agents (e.g. gellan gum, carrageenan, etc.), and HMB, typically as calcium HMB. The resulting CHO-MIN slurry is held for 10 minutes with continued heat and agitation before adding additional minerals (e.g., potassium chloride, magnesium carbonate, potassium iodide, etc.) and/or carbohydrates (e.g., fructooligosaccharide, sucrose, corn syrup, etc.). The PIW slurry is then formed by mixing with heat and agitation the remaining protein (e.g., sodium caseinate, soy protein concentrate, etc.) into water.

The resulting slurries are then blended together with heated agitation and the pH adjusted to the desired range, typically from 6.6-7.0, after which the composition is subjected to high-temperature short-time (HTST) processing during which the composition is heat treated, emulsified and homogenized, and then allowed to cool. Water soluble vitamins and ascorbic acid are added, the pH is again adjusted to the desired range if necessary, flavors are added, and water is added to achieve the desired total solid level. The composition is then aseptically packaged to form an aseptically packaged nutritional emulsion, or the composition is added to retort stable containers and then subjected to retort sterilization to form retort sterilized nutritional emulsions.

The manufacturing processes for the nutritional liquids may be carried out in ways other than those set forth herein without departing from the spirit and scope of the present disclosure. The present embodiments are, therefore, to be considered in all respects illustrative and not restrictive and that all changes and equivalents also come within the description of the present disclosure.

The nutritional solid, such as a spray dried nutritional powder, dry-mixed nutritional powder or combination thereof, may be prepared by any collection of known or otherwise effective techniques suitable for making and formulating a nutritional powder.

For example, when the nutritional powder is a spray dried nutritional powder, the spray drying step may likewise include any spray drying technique that is known for or otherwise suitable for use in the production of nutritional powders. Many different spray drying methods and techniques are known for use in the nutrition field, all of which are suitable for use in the manufacture of the spray dried nutritional powders herein.

One method of preparing the spray dried nutritional powder comprises forming and homogenizing an aqueous slurry or liquid comprising HMB, typically calcium HMB, and optionally protein, carbohydrate, and fat, and then spray drying the slurry or liquid to produce a spray dried nutritional powder. The method may further comprise the step of spray drying, dry mixing, or otherwise adding additional nutritional ingredients, including any one or more of the ingredients described herein, to the spray dried nutritional powder. As noted, the methods of manufacture desirably utilized calcium HMB, which is most typically formulated as calcium HMB monohydrate, as the HMB source for use in the methods.

### Methods of Facilitating Muscle Recovery Using HMB

The nutritional products including the HMB are administered orally in accordance with the present disclosure to an individual as needed to facilitate muscle recovery after muscle disuse or immobilization. The immobilization may be due to any number of reasons, including for example bed rest, hospitalization, casting, weightlessness, inactivity, and the like, as noted above. Although the HMB-containing nutritional product may be administered to an individual, including an older adult, solely during the period of muscle disuse or solely during the period of muscle recovery, to more fully facilitate muscle recovery, it is generally desirable to administer the HMB-containing nutritional product to the individual during at least a portion of both the period of muscle disuse and the period of muscle recovery. In a desirable embodiment, the HMB-containing nutritional product is administered to the individual for the entire, or substantially entire, period of muscle disuse and the entire, or substantially entire, period of muscle recovery. The individual may be an adult or older adult who is susceptible to muscle atrophy due to muscle disuse or immobilization, at risk of muscle atrophy due to muscle disuse or immobilization, or who actually has muscle atrophy due to muscle disuse or immobilization. In some embodiments described herein, the individual is in need of assistance to facilitate muscle recovery after muscle disuse or immobilization. As such, in some embodiments, not all individuals may benefit from the nutritional products and methods of the present disclosure as not all individuals have a need for muscle recovery after muscle disuse or immobilization.

Once the period of muscle disuse is over, the HMB-containing nutritional product may be administered during the period of muscle recovery for a period of at least one week, including at least one month, including at least six months, and including one year or longer to facilitate muscle recovery. In one specific embodiment, the HMB-containing nutritional product is administered for a continuous period of from one week to six months, including one month to six months following the period of muscle disuse. As noted above, the HMB-containing nutritional product may also be administered during a portion or all of the period of muscle disuse.

The methods of the present disclosure are further directed to facilitating muscle recovery in an individual, including adults and older adults, having muscle atrophy caused by a period of muscle disuse. It will be recognized by one skilled in the art that the rate of muscle atrophy for individuals may differ on the basis of, for example age; that is, older adults may experience more rapid muscle atrophy as compared to adults who may experience more rapid muscle atrophy as compared to younger adults or teenagers. The methods of the present disclosure are suitable for use for all of these age categories, irrespective of the rate of muscle atrophy experienced by an individual during a period of muscle disuse. The methods described herein can be used to increase muscle mass of the individual and can also be used to prevent further muscle atrophy typically associated with muscle reloading after extended periods of muscle disuse in the individual. These methods also include the administration of an HMB-containing nutritional product for methods of (1) stimulating protein synthesis to build muscle; (2) reducing or attenuating muscle loss by preventing muscle protein degradation; (3) increasing muscle force after extended periods of muscle disuse; (4) attenuating myonuclear apoptosis induced by muscle disuse; (5) minimizing muscle atrophy in an individual whose muscles have been subject to a period of muscle disuse; (5) facilitating muscle recovery in an older adult having muscle atrophy caused by a period of muscle disuse; and (6) activating of satellite cells to facilitate muscle regeneration and/or recovery.

### EXAMPLES

The following examples illustrate specific embodiments and or features of the present disclosure. The examples are given solely for the purpose of illustration and are not to be construed as limitations of the present disclosure, as many variations thereof are possible without departing from the spirit and scope of the disclosure. All exemplified amounts are weight percentages based upon the total weight of the product, unless otherwise specified.

The exemplified products are calcium HMB-containing nutritional products that may be prepared in accordance with manufacturing methods well known in the nutrition industry for preparing nutritional emulsions and powders and suitable for use in the methods of the present disclosure.

### Example 1

In this Example, the efficacy of HMB to reduce muscle atrophy and promote muscle recovery following muscle disuse in aged animals is evaluated.

Sixty-four aged (34 months of age) Fischer 344× Brown Norway rats are randomly assigned to a hind limb suspended (HS) group or a reloaded (R) group. The rats in each test group receive either: (1) 1 ml Ca-HMB (170 mg/ml distilled water); or (2) 1 ml of distilled water by gavage feeding per day. After one week, thirty-two rats (eight rats from the HS group receiving Ca-HMB; eight rats from the R group receiving Ca-HMB; eight rats from the HS group receiving water; and eight rats from the R group receiving water) are designated as part of the control groups (HS Control Group (16 rats) or R Control Group (16 rats)). The control groups maintain normal mobility throughout the test period, and are allowed to move freely around their cages. Data is collected from the HS Control Group after 14 days from initiation of the study prior to and following sacrifice. Data is collected from the R Control Group after 28 days from initiation of the study prior to and following sacrifice.

The remaining thirty-two rats (16 rats in HS Group and 16 rats in R Group) continue to receive either Ca-HMB or water and are subjected to hind limb suspension for 14 days. For the hind limb suspension, tape is applied along the proximal one-third of the tail and then placed through a wire harness that is attached to a fishlike swivel at the top of a specially designed hind limb suspension cage. The suspension allowed the rats 360° of movement around the cage. Sterile gauze is wrapped around the tape and is subsequently covered with a thermoplastic material. The exposed tip of the tail is monitored to ensure that it remains pink, indicating that suspension does not interfere with blood flow to the tail. The suspension height is monitored and adjusted to prevent contact between the hind limb and any supportive surface of the cage. Further, the suspension angle does not exceed 30°. The forelimbs maintain contact with a grid floor, which allows the animals to move, groom themselves, and obtain food and water freely.

After 14 days of hind limb suspension, the hind limbs are released. Data is collected from the HS Group prior to and following sacrifice. The R Group is further allowed normal cage ambulation for an additional 14 days and then data is collected prior to and following sacrifice.

Data is collected from all test groups to analyze: (1) change in muscle force over the testing period; (2) body weight change over the testing period; (3) muscle mass at the end of the testing period; (4) presence of apoptotic signaling proteins at the end of the test period; and (5) change in muscle fiber (cross-section) at the end of the testing period.

### Experimental Procedures

Force measurements: All force measurements are made while the animals are anesthetized with 98% oxygen and 2% isoflurane gas. The animals are placed supine on a heated X-Y positioning table using a custom built rat dynamometer, with the left foot secured to the footplate at an ankle angle of 90°. Vertical forces are translated to a load cell transducer in the load cell fixture on the footplate. Platinum stimulating electrodes (Grass Medical Instruments, Quincy, Mass.) are inserted subcutaneously to span the tibial nerve in the popliteal fossa. The maximal isometric force of the plantar flexor muscle group is evaluated by stimulating the tibial nerve using supramaximal square wave pulses that are 4 V, 100 Hz for a duration of 3 seconds using a SD9 stimulator (Grass Medical Instruments, Quincy, Mass.). Maximal force is determined using Labview based software. The maximal forces for three isometric contractions are averaged for each data point. Maximal isometric force measurements are made before 14 days of hind limb suspension (day 0), immediately after 14 days of hind limb suspension, and 14 days after-reloading (recovery of) the hind limbs following 14 days of hind limb suspension.

Body Weight and Tissue Preparation:

Each animal is weighed at the beginning of the experiment, following 14 days of hind limb suspension and after 14 days of reloading. With the animals deeply anesthetized, the soleus and the plantaris muscles are removed from both limbs, then blotted, and weighed. Animals are euthanized following this procedure. A block obtained from the mid-belly of the muscle is embedded in optimal cutting temperature (OCT) compound (Tissue-Tek; Andwin Scientific, Addison, Ill.), snap frozen in liquid nitrogen-cooled isopentane, and stored at −80° C. The remainder of the muscle is snap frozen in liquid nitrogen and stored at −80° C. until needed for subsequent analyses.

Identification Of Apoptotic Nuclei

10-μm-thick frozen cross sections from soleus and plantaris muscles are mounted on charged microscope slides (Fisher Scientific, Pittsburgh, Pa.). Apoptotic nuclei are identified by labeling the sections with fluorescent labeling of terminal dUTP nick-end labeling (TUNEL) (11684795910; Roche Applied Science, Indianapolis, Ind.) and lamina. Briefly, the tissue sections are fixed with 4% paraformaldehyde in phosphate buffered saline (PBS) and permeabilized with 0.1% Triton X-100 in PBS. The tissue is then incubated at 4° C. overnight with a rat anti-lamina monoclonal antibody (MAB 1914, Millipore, Billerica, Mass.) to visualize the basal lamina of each muscle fiber. The sections are then incubated with donkey anti-rat rhodamine conjugated second antibody (712-025-150, Jackson ImmunoResearch Laboratories, West Grove, Pa.) along with the TUNEL reaction mixture in a humidified chamber at 37° C. for 1 hour in the dark. Omission of the TdT enzyme in the TUNEL reaction mixture on one of the tissue sections on each slide is included as a negative control. The sections are mounted with 4′,6-diamidino-2-phenylindole (DAPI) to visualize all nuclei (Vectashield mounting medium; Vector Laboratories, Burlingame, Calif.) and viewed under a Zeiss LSM 510 Meta confocal microscope (Carl Zeiss Microimaging Inc. Thornwood, N.Y.). The number of TUNEL- and DAPI-positive nuclei that are immediately adjacent to, or beneath, the basal lamina is counted. Data is expressed as an apoptotic index, which is calculated by counting the number of TUNEL-positive nuclei divided by the total number of nuclei (i.e., DAPI-positive nuclei). The apoptotic index is determined from ˜1200 fibers, which are obtained from four non-overlapping regions of each tissue cross section.

Fiber Morphology:

Muscle fiber cross sectional area (CSA) is determined by planimetry from 750-1200 fibers that are obtained from four non-overlapping regions of each tissue cross-section is stained for lamina. Fiber CSA is calculated by the Image J software.

Western Immunoblots:

Approximately seventy-five micrograms of muscle is homogenized in ice-cold RIPA buffer (1% Triton x-100, 150 mM NaCl, 5 mM EDTA, 10 mM Tris; pH 7.4), containing protease inhibitors (P8340, Sigma-Aldrich, St. Louis, Mo.), and phosphatase inhibitors (P2850; P5726, Sigma-Aldrich). The muscle homogenates are centrifuged at 1000×g for 5 minutes at 4° C., and the protein content of the supernatant is measured (500-0116; BioRad, Hercules, Calif.). Forty micrograms of protein are loaded into each well of a 4-12% gradient polyacrylamide gel (NP0335BOX; Invitrogen, Carlsbad, Calif.) and separated by routine sodium dodecyl sulfate-polyacrylamide gel electrophoresis (SDS-PAGE) for 1 hour at 120V. The proteins are transferred to a nitrocellulose membrane for 1.5 hours at 25V. Non-specific protein binding is blocked by incubating the membranes in 5% nonfat milk in Tris-buffered saline containing 0.05% Tween 20 (TBST) at room temperature. The membranes are incubated (1:1000) overnight at 4° C. with primary antibodies directed against Bcl-2 (#2876, Cell Signaling Technology, Boston, Mass.), Bax (#2772, Cell Signaling), cleaved caspase-3 (#9664, Cell Signaling) and cleaved caspase-9 (#9509, Cell Signaling). The membranes are washed in TBST and incubated in appropriate dilutions of secondary antibodies (diluted in 5% non-fat milk) conjugated to horseradish peroxidase (Sigma-Aldrich, St. Louis, Mo.). The signals are developed using a chemiluminescent substrate (Lumigen TMA-6; Lumigen, Southfield, Mich.) and visualized by exposing the membranes to x-ray films (BioMax MS-1; Eastman Kodak). Digital records are captured with a Kodak 290 camera, and protein bands are quantified using 1D analysis software. The bands are quantified as optical density X band area and expressed in arbitrary units.

BrdU Administration:

A subcutaneous time-released bromodeoxyuridine (BrdU) pellet (21-days release, 0.22 μg BrdU/gm body mass/day, Innovative Research, Sarasota, Fla.) was implanted in each rat at the point of reloading. The animals were anesthetized with 2% isoflurane, and the BrdU pellet was inserted subcutaneously on the dorsum over the upper thoracic spine. BrdU was used to identify activated satellite cells/muscle precursor cells during periods of muscle reloading because BrdU is a thymidine analog and is incorporated in nuclei during DNA synthesis.

Immunofluorescent Staining.

Activated, proliferated satellite cells/muscle precursor cells were identified by immunofluorescence staining on BrdU and laminin as described previously (Siu 2005). Anti-BrdU mouse monoclonal antibody and anti-mouse IgG Cy3 conjugate F(ab′)₂ fragment was used for detection. In order to visualize the fiber basal lamina, anti-rat laminin mouse monoclonal followed by an anti-mouse IgG biotin-conjugated antibody were used. The sections were finally stained with 4′,6-diamidino-2-phenylindole (DAPI) and examined under a fluorescence microscope with excitation wavelengths of 330-380 nm for DAPI blue fluorescence, 485-585 nm for Cy3 red fluorescence, and 450-490 nm for fluorescein green fluorescence. Images were obtained using a SPOT RT camera and software. The numbers of BrdU- and DAPI-positive nuclei were counted from six random non-overlapping fields at an objective magnification of 40×. Only the labeled nuclei that were under the laminin staining were counted in order to exclude any non-muscle nuclei in the sections.

Statistical Analysis:

The results are reported as means±SE. Differences in means between groups are determined by multiple analysis of variance (MANOVA), and Hotelling's T-Square test. Bonferroni post hoc analyses are performed between significant means. Chi-squared analyses are conducted between experimental groups for the fiber area-frequency data. A P value≦0.05 is considered significant.

### Results

Bodyweight:

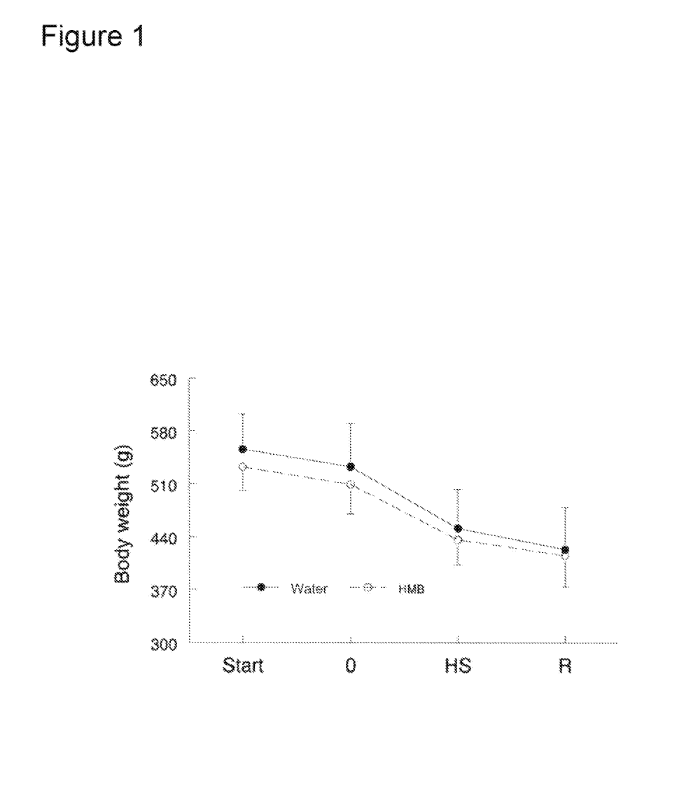

The bodyweight of the animals does not differ among the experimental groups at the beginning of the study. In general, 14 days of hind limb suspension dramatically lowered body weight by about 15% after treatment in both the HMB and water-treated groups. The animal's bodyweight continues to decline during the reloading period in both groups. The body weight of water-treated rats decreases by 1.6% after three weeks (1 week of pre-treatment and 14 days of hind limb suspension), and 9.3% after five weeks (pre-treatment, hind limb suspension and 14 days of reloading), into the experimental protocol, respectively. The body weight of the animals is reduced by 1.3% and 7% in control rats treated with HMB for three and five weeks, respectively, relative to the first experimental day. The animals' body weights continue to decline after 2 weeks of reloading in both HMB and water-treated rats. Overall, there is a 4% decrease in the body weight of HMB-treated rats, and a 6% decrease in the bodyweight of water-treated rats, after 14 days of reloading compared to the body weight of control animals (See FIG. 1).

Maximal Isometric Force:

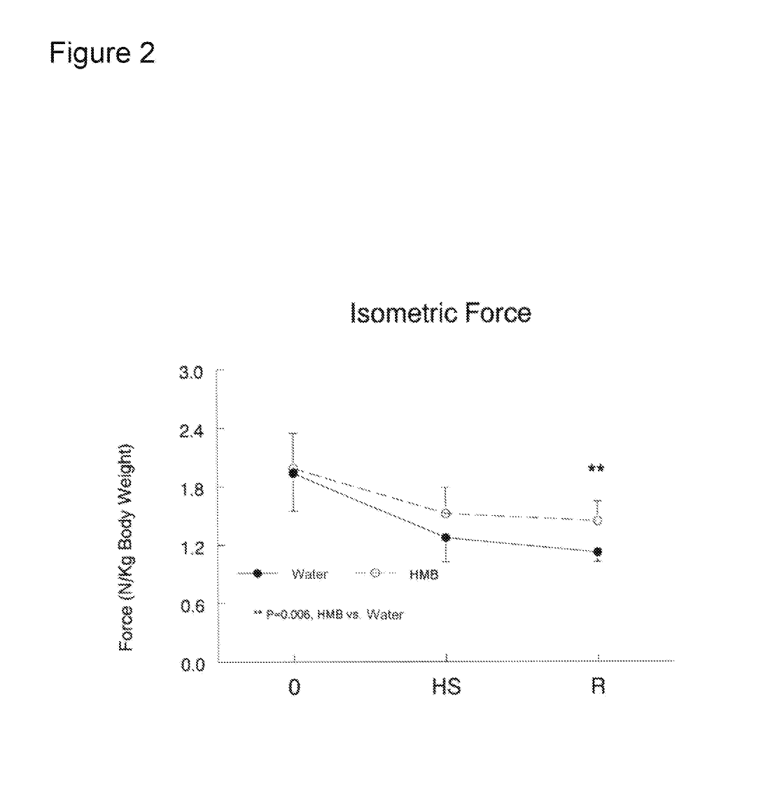

HMB appears to attenuate the loss of force with hind limb suspension and reloading. Maximal isometric force is not different among the groups prior to hind limb suspension. Hind limb suspension reduces maximal in vivo plantarflexor isometric force by 34.3% in water-treated rats, and by 23.7% in HMB-treated rats. There is a greater loss in maximal isometric plantarflexor force of the water-treated rats (42.4%) than HMB-treated animals (27.3%) after reloading as compared to isometric force in control animals (P<0.01, See FIG. 2).

Muscle Wet Weight:

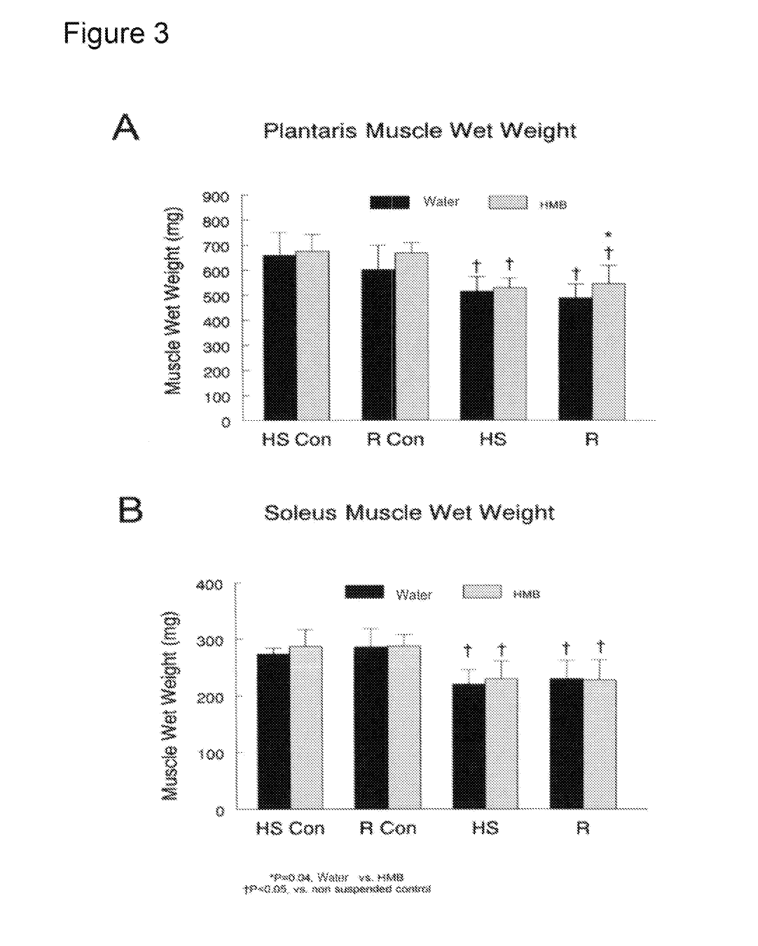

HMB did not significantly reduce the extent of HS-induced atrophy, but it did improve muscle wet weight in the plantaris muscle of the reloaded group, relative to the vehicle control animals. HS induced a significant decrease in the wet weight of the plantaris (19%) and soleus (15%) muscles (P<0.001), of both HMB and vehicle-treated animals (n=16 per group). There was no significant difference in the extent of muscle mass loss between the experimental groups after 14 days of HS. Reloading prevented any further declines in soleus and plantaris muscle wet weight, but it did not reverse the muscle loss induced by HS. HMB treatment significantly improved plantaris muscle weight after 14 days of reloading relative to vehicle-treated animals (n=8 per group) (FIG. 3A). HMB did not provide protective effect against HS-induced loss in the soleus muscle, nor did it improve soleus muscle wet weight recovery after 14 days of reloading, relative to the vehicle-treated animals (FIG. 3B).

Changes of Muscle Fiber CSA:

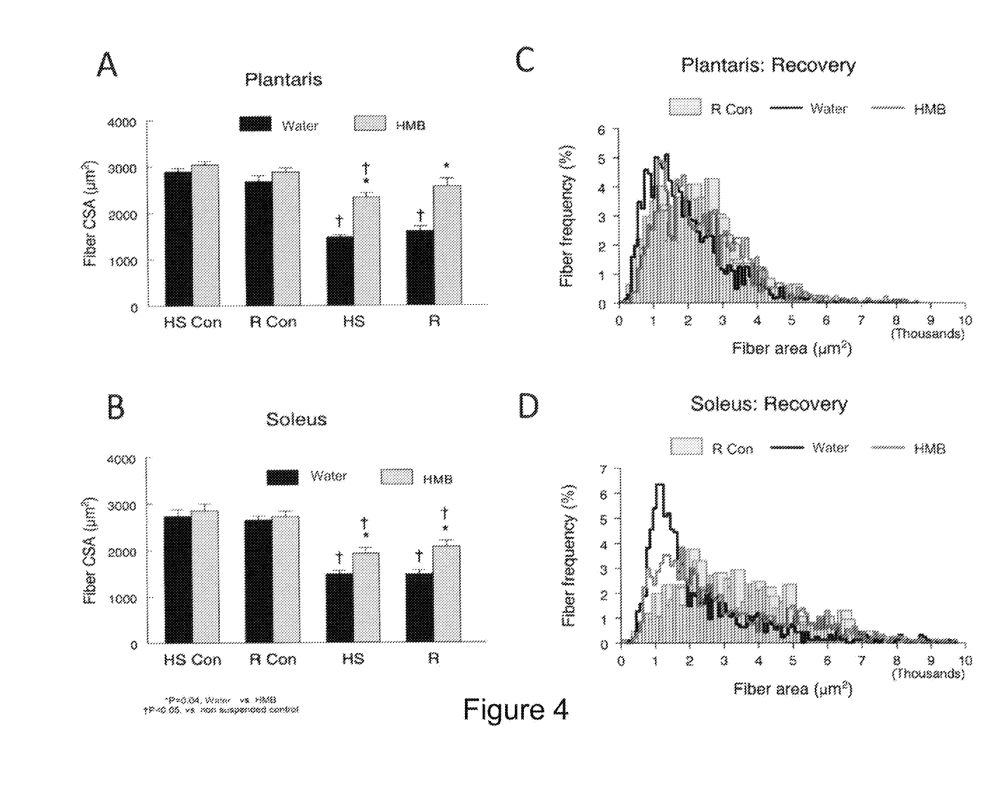

HMB reduced the extent of fiber atrophy in both soleus and plantaris muscles that occurred after HS or reloading. HS dramatically decreased mean fiber CSA in both plantaris (FIG. 4A) and soleus (FIG. 4B) hindlimb muscles. However, the HS-induced decrease in fiber CSA of the vehicle-treated animals was greater than in the HMB-treated animals for plantaris (48.8% vs. 26.4%, p<0.05) and soleus muscles (45.6% vs. 32.5%, p<0.05). HMB did not further improve fiber CSA in either plantaris (FIG. 4A) or soleus (FIG. 4B) muscles after 14 days of reloading as compared to HS. The fiber area-fiber frequency distribution for the plantaris (FIG. 4C) and soleus (FIG. 4D) are shown for the recovery group. After 14 days of recovery, the muscle fibers in both the plantaris and soleus were still shifted to the left in the fiber area-frequency distribution, relative to the distribution of the control muscles. Chi-squared analyses showed that the frequency of fibers<1500 μm² was significantly greater in the plantaris and soleus muscles of vehicle-treated animals as compared to HMB-treated animals that had recovered for 14 days following HS. The distribution of the fiber area-frequency distribution after HS (FIG. 4C, FIG. 4D) was similar to that shown for recovery (data not shown).

Apoptotic Myonuclei as Identified by TUNEL Labeling:

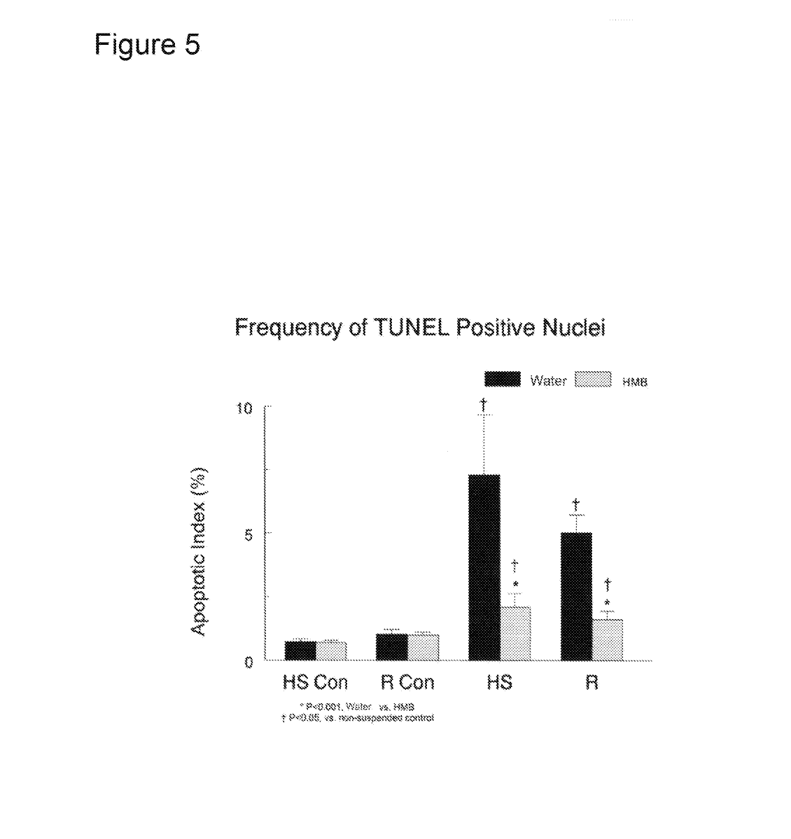

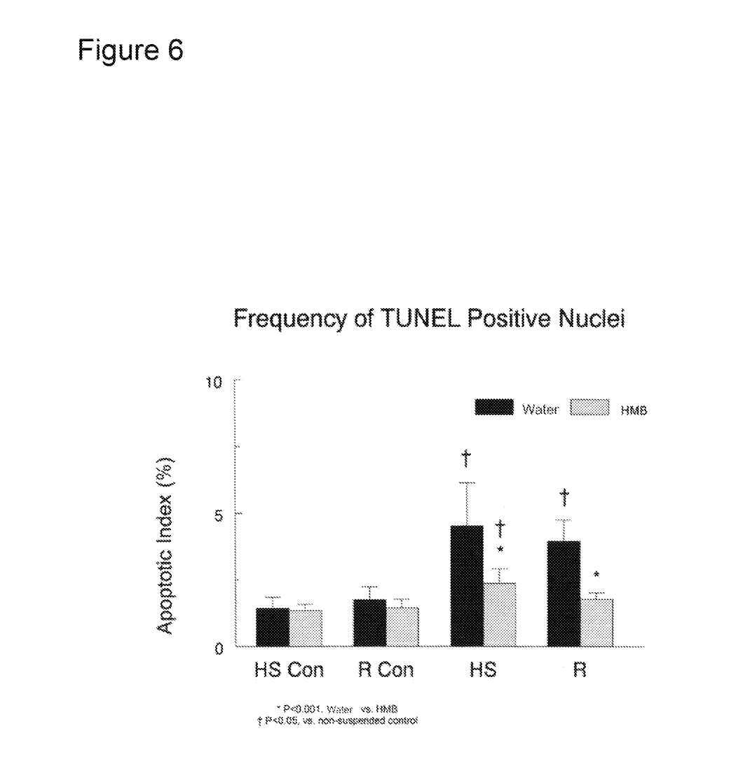

The apoptotic index as indicated by the frequency of TUNEL positive myonuclei, was markedly elevated by HS, and reloading, and HMB attenuated the apoptotic index. HS significantly increased TUNEL positive nuclei in both plantaris and soleus muscles. Although there were some regional differences within tissue sections, TUNEL-positive nuclei occurred throughout each tissue cross section that was obtained from plantaris and soleus muscles following HS. The apoptotic index was significantly increased in plantaris (9.9 fold, p<0.05) and soleus (3.2 fold p<0.05) muscles of vehicle-treated animals as compared to ambulatory control animals. Although HMB treatment suppressed myonuclear apoptosis, it did not completely eliminate it. HS increased the apoptotic index in both plantaris (3.0 fold p<0.05) and soleus (1.8 fold p<0.05) muscles of HMB-treated animals compared to ambulatory control animals. The apoptotic index was significantly greater (p<0.001) in both plantaris (FIG. 5) and soleus muscles from vehicle as compared to HMB-treated animals (FIG. 6). Apoptosis, as identified by TUNEL labeling, remained high during reloading, especially in vehicle-treated muscles. Similar to the results after HS, HMB continued to suppress TUNEL labeling in the myonuclei of reloaded plantaris (FIG. 5) and soleus (FIG. 6) muscles. Nevertheless, there was no significant difference between the apoptotic indexes of muscles, after HS, as compared to reloaded conditions within either the water- or HMB-treated groups.

Apoptotic Signaling Proteins:

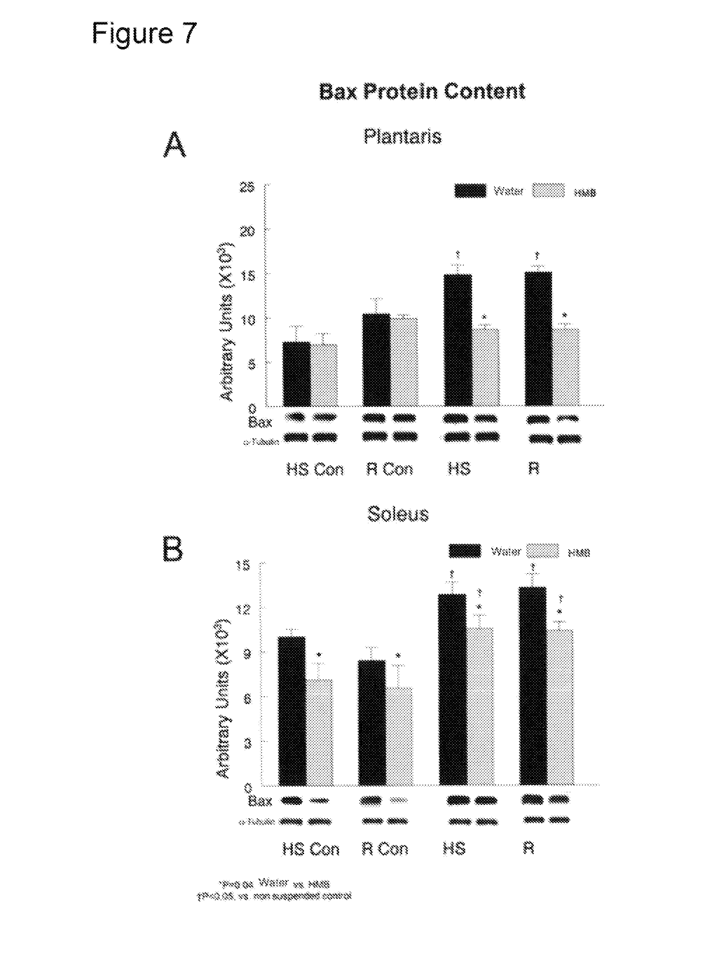

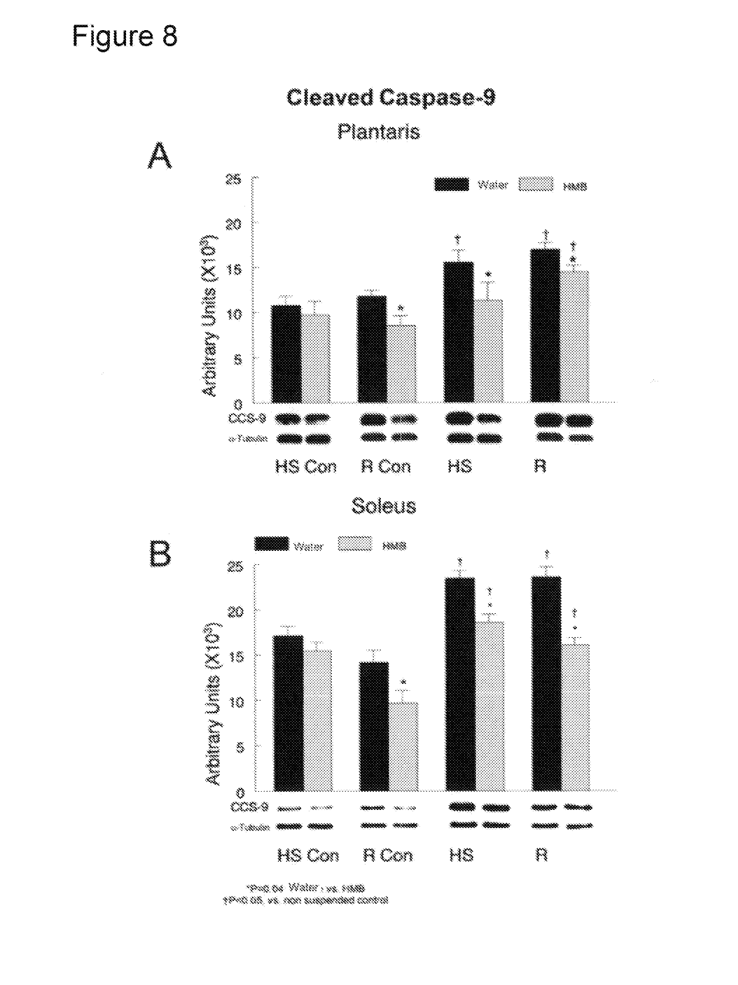

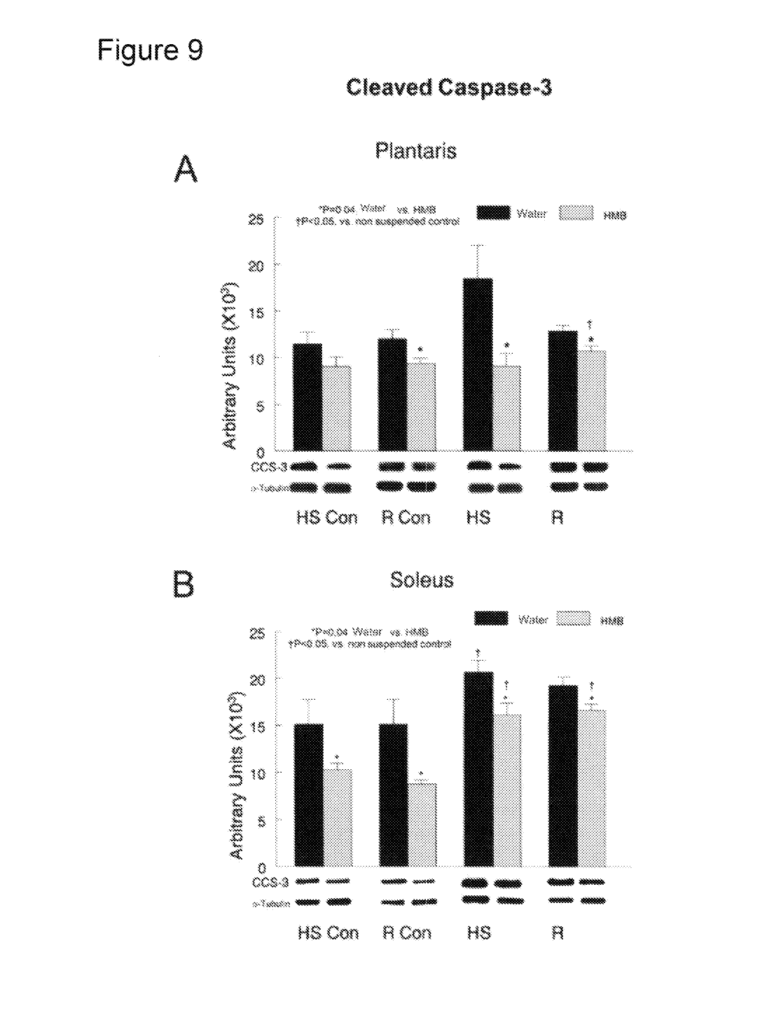

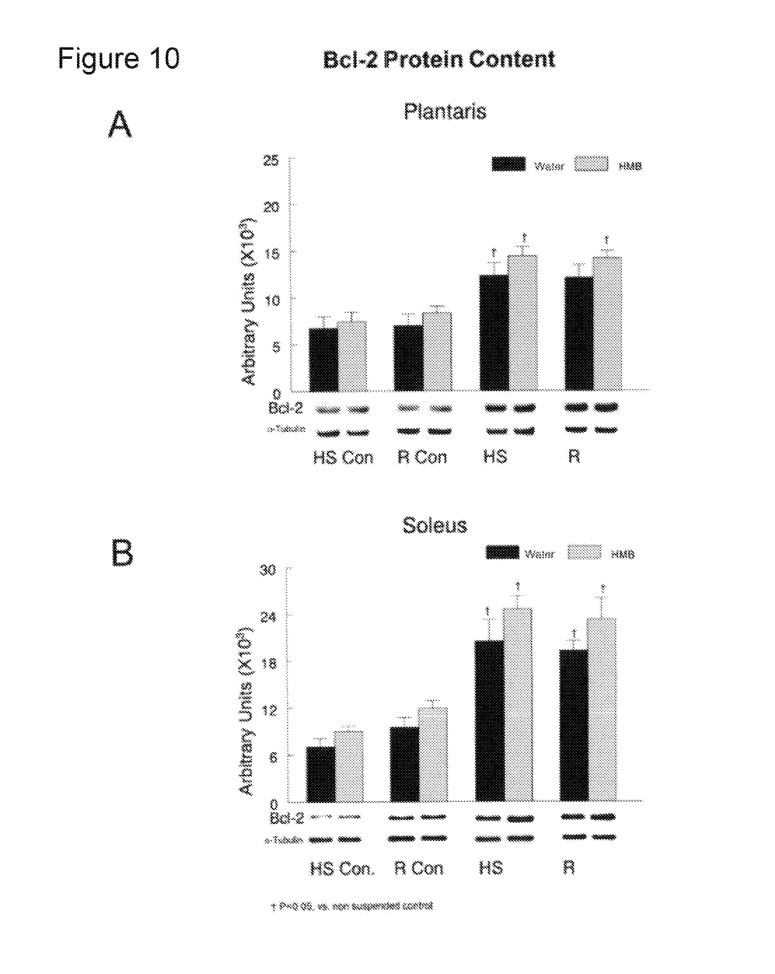

HMB treatment suppresses the hind limb suspension-induced increase in the pro-apoptotic proteins after hind limb suspension and reloading, as compared to water-treated rats. Pro-apoptotic proteins associated with mitochondrial apoptotic signaling increases in abundance in hind limb muscles after hind limb suspension, and remains elevated during reloading. Pro-apoptotic proteins including Bax (FIG. 7), cleaved caspase-9 (FIG. 8), and cleaved caspase-3 (FIG. 9) increases after both hind limb suspension and reloading. HMB suppresses the protein abundance for Bax (FIG. 7), cleaved caspase-9 (FIG. 8), and cleaved caspase-3 (FIG. 9) in plantaris and soleus muscles after both hind limb suspension and reloading conditions. The abundance of pro-apoptotic signaling proteins is similar in the plantaris (FIG. 7A, 8A, 9A) and soleus (FIG. 7B, 8B, 9B) muscles after hind limb suspension and reloading, and this is not altered by HMB. HMB reduces the protein abundance of Bax (FIG. 7B), and cleaved caspase-3 (FIG. 9B) in control muscles of ambulatory animals (HS Con and R Con). Both hind limb suspension and reloading increase the anti-apoptotic protein, Bcl-2, in plantaris (FIG. 10A) and soleus (FIG. 10B) muscles by about 100% (P<0.05), but there may not be a significant difference between HMB and water-treated muscles after either hind limb suspension or reloading.

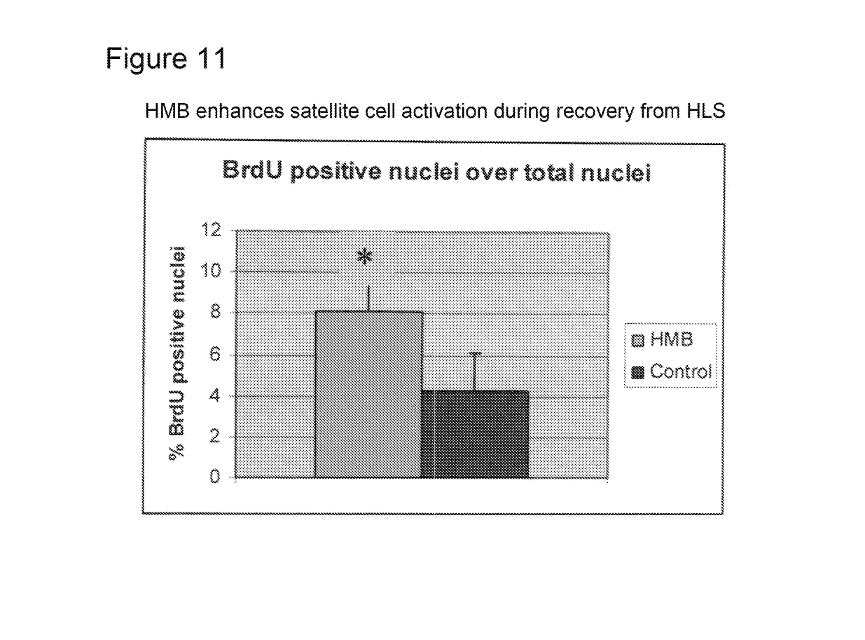

HMB treatment resulted in the activation of satellite cells, which are muscle precursor cells required for muscle repair and regeneration. BrdU labeling identifies new myonuclei within existing myofiber that are generally obtained from fusion of an activated and differentiated satellite cell into existing myofibers. This is a natural process of muscle repair and regeneration. During recovery from muscle disuse, the HMB-treated group had almost twice the number of BrdU positive nuclei than vehicle-treated rats (HMB=8.1+/−2; Control=4.2+/−1.7 (p=0.001). FIG. 11 shows results of the immunofluorescent labeling of BrdU and laminin that was used to identify the BrdU-positive nuclei in the soleus as an estimate of the activated/proliferating muscle satellite cell nuclei. BrdU was stained with secondary Cy3 laminin stained with secondary fluorescein and nuclei labeled with DAPI. The number of BrdU-positive nuclei to total nuclei was expressed as a percent. More than 2500 nuclei were assessed per group. As noted above, the HMB group showed almost twice the number of BrdU-positive nuclei over the 14 day recovery period, indicating early signs of muscle hypertrophy due to activation of satellite cells in the treatment group.

### Analysis of Results

As the data above indicate, HMB improves muscle recovery following hind limb suspension and subsequent reloading. Additionally, HMB: (1) prevents further force loss during reloading (recovery) after unloading (immobilization); (2) improves muscle mass in the plantaris of reloaded muscles; (3) blunts the extent of fiber atrophy in both fast and slow skeletal muscles in response to unloading and reloading; (4) significantly attenuates myonuclear apoptosis induced by HS; (5) decreases the apoptotic index after reloading in both plantaris and soleus muscles; and (6) reduces mitochondrial apoptotic signaling as indicated by lower levels of cleaved caspase-3, cleaved caspase-9 and Bax protein abundance in reloaded plantaris and soleus muscles. These results indicate that HMB is highly effective in facilitating muscle recovery following a period of muscle disuse.

### Examples 2-6

Examples 2-6 illustrate HMB-containing nutritional powders suitable for use in the methods of the present disclosure, the ingredients of which are listed in the table below. These products are prepared by spray drying methods in separate batches, are reconstituted with water prior to use to the desired target ingredient concentrations. All ingredient amounts are listed as kg per 1000 kg batch of product, unless otherwise specified.

| Ingredient | Ex. 2 | Ex. 3 | Ex. 4 | Ex. 5 | Ex. 6 |
|---|---|---|---|---|---|
| Maltodextrin | 436.7 | 436.7 | 436.7 | 436.7 | 436.7 |
| Sucrose | 145.5 | 145.5 | 145.5 | 145.5 | 145.5 |
| Calcium Caseinate | 129.1 | 129.1 | 129.1 | 129.1 | 129.1 |
| Isolated Soy Protein | 57.7 | 57.7 | 57.7 | 61.7 | 57.7 |
| FOS Powder | 33.6 | 33.6 | 33.6 | 33.6 | 32.6 |
| HO sunflower oil | 59.9 | 55.5 | 61.24 | 57.2 | 62.58 |
| Calcium HMB | 31.6 | 34.6 | 28.6 | 27.6 | 32.6 |
| Canola Oil | 55.1 | 53.7 | 56.4 | 52.42 | 57.78 |
| Soy Oil | 26.7 | 26.0 | 27.37 | 25.36 | 28.04 |
| Potassium Citrate | 10.3 | 10.3 | 10.3 | 10.3 | 10.3 |
| Sodium Citrate | 5.8 | 5.8 | 5.8 | 5.8 | 5.8 |
| Potassium Chloride | 5.2 | 5.2 | 5.2 | 5.2 | 5.2 |
| Magnesium Chloride | 4.7 | 4.7 | 4.7 | 4.7 | 4.7 |
| Potassium hydroxide | 3.1 | 3.1 | 3.1 | 3.1 | 3.1 |
| Sodium phosphate dibasic dihydrate | 3.0 | 3.0 | 3.0 | 3.0 | 3.0 |
| Sodium chloride | 2.5 | 2.5 | 2.5 | 2.5 | 2.5 |
| Choline Chloride | 1.8 | 1.8 | 1.8 | 1.8 | 1.8 |
| Vanilla Flavor | 1.8 | 1.8 | 1.8 | 1.8 | 1.8 |
| Sodium phosphate monobasic monohydrate | 1.6 | 1.6 | 1.6 | 1.6 | 1.6 |
| Potassium phosphate dibasic trihydrate | 1.1 | 1.1 | 1.1 | 1.1 | 1.1 |
| Flavor | 1.0 | 1.0 | 1.0 | 1.0 | 1.0 |
| Vitamin premix | 1.0 | 1.0 | 1.0 | 1.0 | 1.0 |
| Ascorbyl palmitate | 0.243 | 0.243 | 0.243 | 0.243 | 0.243 |
| Ascorbic acid | 0.240 | 0.240 | 0.240 | 0.240 | 0.240 |
| Antioxidant | 0.116 | 0.116 | 0.116 | 0.116 | 0.116 |
| Ferrous sulfate | 0.010 | 0.090 | 0.030 | 0.020 | 0.070 |
| Vitamin premix | 0.065 | 0.065 | 0.065 | 0.065 | 0.065 |
| Zinc sulfate monohydrate | 0.057 | 0.057 | 0.057 | 0.057 | 0.057 |
| Manganese sulfate | 0.045 | 0.045 | 0.045 | 0.045 | 0.045 |
| Mineral mix copper sulfate | 0.035 | 0.035 | 0.035 | 0.035 | 0.035 |
| Beta carotene | 0.005 | 0.005 | 0.005 | 0.005 | 0.005 |
| Chromium chloride | 0.001 | 0.001 | 0.001 | 0.001 | 0.001 |
| Sodium molybdate | 0.0012 | 0.0012 | 0.0012 | 0.0012 | 0.0012 |
| Potassium iodide | 0.001 | 0.001 | 0.001 | 0.001 | 0.001 |
| Sodium selenite | 0.0004 | 0.0004 | 0.0004 | 0.0004 | 0.0004 |
| Citric acid | AN | AN | AN | AN | AN |
| Potassium hydroxide | AN | AN | AN | AN | AN |
| Magnesium sulfate dry | AN | AN | AN | AN | AN |
| Ultra micronized tricalcium phosphate | AN | AN | AN | AN | AN |
| Ascorbic acid | AN | AN | AN | AN | AN |

AN = As Needed

### Examples 7-11

Examples 7-11 illustrate HMB-containing nutritional liquids suitable for use in the methods of the present disclosure, the ingredients of which are listed in the table below. All amounts are listed as kilogram per 1000 kilogram batch of product, unless otherwise specified.

| Ingredient | Ex. 7 | Ex. 8 | Ex. 9 | Ex. 10 | Ex. 11 |
|---|---|---|---|---|---|
| Water | Q.S. | Q.S. | Q.S. | Q.S. | Q.S. |
| Sucrose | 89.3 | 89.3 | 89.3 | 89.3 | 89.3 |
| Maltodextrin | 29.7 | 29.7 | 29.7 | 29.7 | 29.7 |
| Sodium Caseinate | 25.9 | 25.9 | 25.9 | 25.9 | 25.9 |
| Milk Protein Concentrate | 19.1 | 19.1 | 19.1 | 19.1 | 19.1 |
| Soy Protein Isolate | 11.9 | 11.9 | 9.9 | 12.9 | 13.9 |
| Potassium Citrate | 7.9 | 7.9 | 7.9 | 7.9 | 7.9 |
| Soy Oil | 11.1 | 9.9 | 11.4 | 10.7 | 11.6 |
| Calcium HMB | 6.7 | 7.7 | 8.7 | 5.7 | 4.7 |
| Canola Oil | 10.2 | 10.0 | 10.5 | 9.8 | 10.7 |
| Corn Oil | 9.3 | 9.1 | 9.6 | 8.9 | 9.8 |
| Whey Protein Concentrate | 3.5 | 3.5 | 3.5 | 3.5 | 3.5 |
| Magnesium Phosphate Dibasic | 3.1 | 3.1 | 3.1 | 3.1 | 3.1 |
| Flavoring Agent | 2.0 | 2.0 | 2.0 | 2.0 | 2.0 |
| Stabilizer | 2.0 | 2.0 | 2.0 | 2.0 | 2.0 |
| Soy Lecithin | 1.5 | 1.5 | 1.5 | 1.5 | 1.5 |
| Sodium Phosphate Dibasic Dihydrate | 1.3 | 1.3 | 1.3 | 1.3 | 1.3 |
| Potassium Phosphate Dibasic | 0.985 | 0.985 | 0.985 | 0.985 | 0.985 |
| Potassium Chloride | 0.729 | 0.729 | 0.729 | 0.729 | 0.729 |
| Choline Chloride | 0.480 | 0.480 | 0.480 | 0.480 | 0.480 |
| Ascorbic Acid | 0.469 | 0.469 | 0.469 | 0.469 | 0.469 |
| Calcium Carbonate | 0.451 | 0.451 | 0.451 | 0.451 | 0.451 |
| Caramel Flavor | 0.450 | 0.450 | 0.450 | 0.450 | 0.450 |
| Dairy Creamer | 0.450 | 0.450 | 0.450 | 0.450 | 0.450 |
| UTM/TM Premix | 0.367 | 0.367 | 0.367 | 0.367 | 0.367 |
| 45% Potassium Hydroxide | 0.323 | 0.323 | 0.323 | 0.323 | 0.323 |
| Carrageenan | 0.200 | 0.200 | 0.200 | 0.200 | 0.200 |
| Water Soluble Vitamin Premix | 0.185 | 0.185 | 0.185 | 0.185 | 0.185 |
| Vitamin DEK Premix | 0.067 | 0.067 | 0.067 | 0.067 | 0.067 |
| Sodium Chloride | 0.060 | 0.060 | 0.060 | 0.060 | 0.060 |
| Gellan Gum | 0.050 | 0.050 | 0.050 | 0.050 | 0.050 |
| Vitamin A Palmitate | 0.0082 | 0.0082 | 0.0082 | 0.0082 | 0.0082 |
| Corn oil carrier | Q.S. | Q.S. | Q.S. | Q.S. | Q.S. |
| Vitamin D₃ | 399 mg | 399 mg | 399 mg | 399 mg | 399 mg |
| Potassium Iodide | 194 mg | 194 mg | 194 mg | 194 mg | 194 mg |

What is claimed is:

1. A method for facilitating muscle recovery in an individual having muscle atrophy caused by a period of muscle disuse, the method comprising administering to the individual during the period of muscle disuse and during a period of muscle recovery a composition comprising an effective amount of beta-hydroxy-beta-methylbutyrate.

2. The method of claim 1 wherein the individual is administered beta-hydroxy-beta-methylbutyrate daily during both the period of muscle disuse and the period of muscle recovery.

3. The method of claim 2 wherein the individual is administered beta-hydroxy-beta-methylbutyrate for a time of at least one month following muscle disuse.

4. The method of claim 2 wherein the individual is administered beta-hydroxy-beta-methylbutyrate for a time of from one month to six months following muscle disuse.

5. The method of claim 2 wherein the individual is administered beta-hydroxy-beta-methylbutyrate for a time of about one year.

6. The method of claim 1 wherein the individual is administered from about 0.1 g/day to about 10 g/day of beta-hydroxy-beta-methylbutyrate during both the period of muscle disuse and the period of muscle recovery.

7. A method for minimizing muscle atrophy in an individual whose muscles have been subject to a period of muscle disuse, the method comprising administering to the individual during the period of muscle disuse and during a period of muscle recovery a composition comprising an effective amount of beta-hydroxy-beta-methylbutyrate.

8. The method of claim 7 wherein the muscles have been in disuse for one week or longer.

9. The method of claim 7 wherein the muscles have been in disuse for a period of one week to one year.

10. The method of claim 7 wherein the individual is administered beta-hydroxy-beta-methylbutyrate daily during both the period of muscle disuse and the period of muscle recovery.

11. The method of claim 10 wherein the individual is administered beta-hydroxy-beta-methylbutyrate for a time of at least one month following the period of muscle disuse.

12. The method of claim 10 wherein the individual is administered beta-hydroxy-beta-methylbutyrate for a period of from one month to six months following the period of muscle disuse.

13. The method of claim 7 wherein the individual is administered from about 0.1 g/day to about 10 g/day of beta-hydroxy-beta-methylbutyrate during both the period of muscle disuse and the period of muscle recovery.

14. A method for facilitating muscle recovery in an older adult having muscle atrophy caused by a period of muscle disuse, the method comprising administering to the older adult during the period of muscle disuse and during a period of muscle recovery a composition comprising an effective amount of beta-hydroxy-beta-methylbutyrate.

15. The method of claim 14 wherein the older adult is administered beta-hydroxy-beta-methylbutyrate daily during both the period of muscle disuse and the period of muscle recovery.
